(19) United States
(12) Patent Application Publication
(10) Pub. No.: US 2024/0271781 A1
(43) Pub. Date: Aug. 15, 2024

(54) CAPTURE METHOD AND CAPTURE STATION FOR FLUE GAS FROM CHEMICAL INDUSTRY PARK, AND APPLICATION THEREOF

(71) Applicant: Shihezi University, Shihezi (CN)

(72) Inventors: Zhaomin LI, Shihezi (CN); Xueqin LI, Shihezi (CN); Haojie LI, Shihezi (CN); Zexin WANG, Shihezi (CN); Xiaoli ZHUANG, Shihezi (CN)

(21) Appl. No.: 18/648,186

(22) Filed: Apr. 26, 2024

(30) Foreign Application Priority Data
Dec. 14, 2023 (CN) 202311716623.4

(51) Int. Cl. F23J 15/00; F23J 15/02

(52) U.S. Cl. CPC F23J 15/006; F23J 15/025

(57) ABSTRACT

A capture method for flue gas from a chemical industrial park is performed as follows. A flue gas is analyzed to obtain composition and ingredient content, and then pre-processed. Classified capture is performed on ingredients of a pre-processed flue gas to obtain a residual tail gas and semi-processed products. Specifically, A classified capture model is constructed and trained, and the analysis data of the pre-processed flue gas is substituted to the trained model to obtain a classified distribution result, based on which the flue gas is distributed into the capture modules according to category for classified capture.

### CROSS-REFERENCE TO RELATED APPLICATIONS

This application claims the benefit of priority from Chinese Patent Application No. 202311716623.4, filed on Dec. 14, 2023. The content of the aforementioned application, including any intervening amendments thereto, is incorporated herein by reference in its entirety.

### TECHNICAL FIELD

This application relates to flue gas treatment technology, and more particularly to a capture method and capture station for flue gas from a chemical industrial park, and an application thereof.

### BACKGROUND

In recent years, with the rapid progress and development of society and industry, the problem of global warming has gradually been known and focused on. The global climate situation has been deteriorating, and the climate crisis has become a problem that cannot be ignored. Carbon dioxide (CO₂), as one of the main greenhouse gases, its emission has been widely concerned around the world. As the largest CO₂ emitter around the world, China's efforts for emission reduction have great significance to global climate governance. The CO₂ emission of China has gradually risen since 1970. After China's accession to the world trade organization (WTO), the CO₂ emission and the annual growth rate of China have increased significantly. Until 2011, with the increase of environmental awareness, the introduction and implementation of relevant policies, and the development of energy-saving and emission reduction technologies, the annual CO₂ emission growth of China began to decline rapidly, and even reached a negative growth rate in 2015 and 2016, which has made a considerable progress to a certain extent. In addition, the annual CO₂ emission growth of European Union and North America have been gradually decreasing since around 2008, the growth rate has remained in the negative range, which are also worthy of recognition for their efforts for emission reduction. China has made a commitment of “PAS 2060-Carbon neutrality” to the international community, which will promote the negative growth of CO₂ emission in the future. It means that China will adopt more strict emission reduction measures, promote the adjustment of energy structure, and strengthen the protection and restoration of the ecosystem to achieve the carbon neutrality goal. This policy background has an important significance for carbon dioxide research.

At present, carbon capture, utilization and storage (CCUS) technique is an effective approach to solve the CO₂ problem commonly used in the world. The CCUS is an industrial process that separating CO₂ from industrial emission sources to directly use or store, so as to achieve CO₂ emission reduction. Specifically, CCUS includes three steps of carbon capture, storage and re-utilization, which can be applied in the emission process of CO₂ generated by large power plants, steel plants, chemical plants and other emission sources. And the CO₂ is collected and stored to avoid emission into the atmosphere, and finally the stored CO₂ is rationally utilized.

Traditional flue gas treatment equipment is to effectively treat the toxic and harmful substances in the flue gas below specified concentration, and to avoid adverse phenomena such as corrosion or blockage of the device. Generally, the flue gas treatment equipment used in incineration plants is divided into dust removal equipment and acid gas removal equipment. Now, with the improvement of people's requirements for environmental quality, most incineration plants have added activated carbon adsorption equipment for heavy metals in the flue gas treatment process.

The CO₂ capture has been widely applied in the flue gas from the chemical industrial parks in recent years, but less attention has been paid to other polluting gases such as nitrogen dioxide (NO₂) and sulfur dioxide (SO₂) in the traditional flue gas treatment processes. With the development of economy and technology, it is necessary to comprehensively consider the capture of CO₂, NO₂ and SO₂, and how to achieve the reasonable and effective capture and distribution of ingredients in the flue gas capture has been a challenge. At present, there is a lack of a scientific and effective distribution and capture method, resulting in poor capture effect and efficiency.

### SUMMARY

This application is to provide a capture method for flue gas from chemical industrial park to solve a problem of unsatisfactory effect and efficiency of capture caused by unreasonable distribution of each ingredient in a flue gas capture process of the chemical industrial park, which realizes accurate calculation of flue gas capture distribution in the chemical industrial park, improves rationality of the flue gas capture distribution, and improves effect and efficiency of capture.

This application provides a capture method for flue gas from a chemical industrial park, comprising:
detecting a flue gas sample in a flue to obtain basic parameters of the flue gas sample comprising composition and content;
pre-processing the flue gas sample;
performing classified capture on ingredients of a pre-processed flue gas to obtain a residual tail gas and a plurality of semi-processed products;
further processing the residual tail gas; and
processing the plurality of semi-processed products to obtain a plurality of processed products.

In an embodiment, the step of detecting a flue gas sample in a flue to obtain basic parameters of the flue gas sample comprises:
detecting and obtaining a flow rate and a pressure of the flue gas sample;
according to the flow rate and the pressure of the flue gas sample, arranging a sampling hole and a detecting module on the flue; and
obtaining the composition and the content of ingredients of the flue gas sample through calculation.

In an embodiment, the step of pre-processing the flue gas sample comprises:
preparing for secondary combustion for the flue gas sample;
performing the secondary combustion on the flue gas sample to obtain a completely-burned flue gas; and
filtering the completely-burned flue gas to obtain the pre-processed flue gas.

In an embodiment, the step of performing classified capture on ingredients of a pre-processed flue gas to obtain a residual tail gas and a plurality of semi-processed products comprises:

constructing a capture module group, wherein the capture module group comprises a plurality of capture modules;

distributing the pre-processed flue gas into the plurality of capture modules according to ingredient category for test capture;

monitoring the plurality of capture modules to obtain test capture data;

based on the test capture data, constructing a classified capture model, and training the classified capture model to obtain a trained classified capture model;

analyzing the pre-processed flue gas to obtain relevant data of the pre-processed flue gas;

substituting the relevant data of the pre-processed flue gas into the trained classified capture model for calculation to obtain a classified distribution result; and

based on the classified distribution result, distributing the pre-processed flue gas into the plurality of capture modules according to the ingredient category for classified capture.

In an embodiment, the test capture data is unifiedly collected as:

$$[ {W}_{XC} , {W}_{XN} , {W}_{XS} , {W}_{XT} , {W}_{XP} , {W}_{QC} , {W}_{QN} , {W}_{QS} , {W}_{VC} , {W}_{VN} , {W}_{VS} ] ;$$

wherein W_{XC} represents a carbon dioxide (CO₂) concentration; W_{XN} represents a nitrogen dioxide (NO₂) concentration; W_{XS} represents a sulfur dioxide (SO₂) concentration; W_{XR} represents temperature; W_{XP} represents pressure; W_{QC} represents a flow distributed to a CO₂ capture module in the test capture; W_{QN} represents a flow distributed to a NO₂ capture module in the test capture; W_{QS} represents a flow distributed to a SO₂ capture module in the test capture; W_{VC} represents CO₂ capture efficiency; W_{VN} represents NO₂ capture efficiency; and W_{VS} represents SO₂ capture efficiency.

The classified capture model is expressed as follows:

$$\{ \text{} \begin{matrix}{V}_{QC} = {F}_{C} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} ) \\ {V}_{QN} = {F}_{N} ( {W}_{XC} , {W}_{XN} , {W}_{XS} , {W}_{XT} , {W}_{XP} , {W}_{QC} , \\ {W}_{QN} , {W}_{QS} , {W}_{VC} , {W}_{VN} , {W}_{VS} ) ; \\ {V}_{QS} = {F}_{S} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} )\end{matrix}$$

wherein V_{QC} represents a calculated flow rate distributed to the CO₂ capture module; V_{QN} represents a calculated flow rate distributed to the NO₂ capture module; and V_{QS} represents a calculated flow rate distributed to the SO₂ capture module;

F_{C}( ) represents a CO₂ capture-calculation model; F_{N}( ) represents a NO₂ capture-calculation model; and FS( ) represents a SO₂ capture-calculation model.

In an embodiment, the step of processing the residual tail gas comprises:
detecting the residual tail gas, and setting a discharge condition;
determining whether the residual tail gas meets the discharge condition according to a detection result; if yes, discharging the residual tail gas; otherwise, further processing the residual tail gas, and repeating above steps until the residual tail gas meets the discharge condition, and discharging the residual tail gas.

In an embodiment, the step of processing the plurality of semi-processed products to obtain a plurality of processed products comprises:
purifying the plurality of semi-processed products; and
compressing a plurality of purified semi-processed products for tank filling.

In another aspect, this application provides a capture station for flue gas from chemical industrial park, comprising:
a detection module;
a pre-processing unit;
the capture module group;
a classified distribution unit;
a first processing unit; and
a second processing unit;
wherein the detection module is configured to detect the flue gas; the pre-processing unit is configured to pre-process the flue gas; the capture module group is configured for classified capture of the flue gas; the classified distribution unit is configured for classified distribution of the flue gas; the first processing unit is configured to process the residual tail gas; and the second processing unit is configured to process the plurality of semi-processed products to obtain the plurality of processed products.

In another aspect, this application provides an application of the capture station in the treatment of flue gas from a chemical industrial park or a kiln.

The present disclosure has at least the following technical effects and advantages.

The present disclosure provides a capture method, capture station and application for flue gas from a chemical industrial park, which solves a problem of unsatisfactory effect and low efficiency of capture caused by unreasonable distribution of each ingredient in a flue gas capture process of the chemical industrial park, realizes accurate calculation of flue gas capture distribution in the chemical industrial park, optimizes capture rationality and capture effects.

### BRIEF DESCRIPTION OF THE DRAWINGS

FIG. 1 is a system block diagram of a capture station for flue gas according to an embodiment of the present disclosure.

FIG. 2 schematically shows an arrangement of a flue and a sampling hole according to an embodiment of the present disclosure.

FIG. 3 is a flow chart diagram of a capture method for flue gas from chemical industrial park according to an embodiment of the present disclosure.

FIG. 4 is a flow chart diagram of the capture method for flue gas from chemical industrial park according to an embodiment of the present disclosure.

FIG. 5 is a flow chart diagram of the capture method for flue gas from chemical industrial park according to an embodiment of the present disclosure.

FIG. 6 is a flow chart diagram of the capture method for flue gas from chemical industrial park according to an embodiment of the present disclosure.

FIG. 7 is a flow chart diagram of the capture method for flue gas from chemical industrial park according to an embodiment of the present disclosure.

FIG. 8 is a flow chart diagram of the capture method for flue gas from chemical industrial park according to an embodiment of the present disclosure.

### DETAILED DESCRIPTION OF EMBODIMENTS

To facilitate the understanding of the above technical solutions, the present disclosure will be further described below with reference to the accompanying drawings and embodiments.

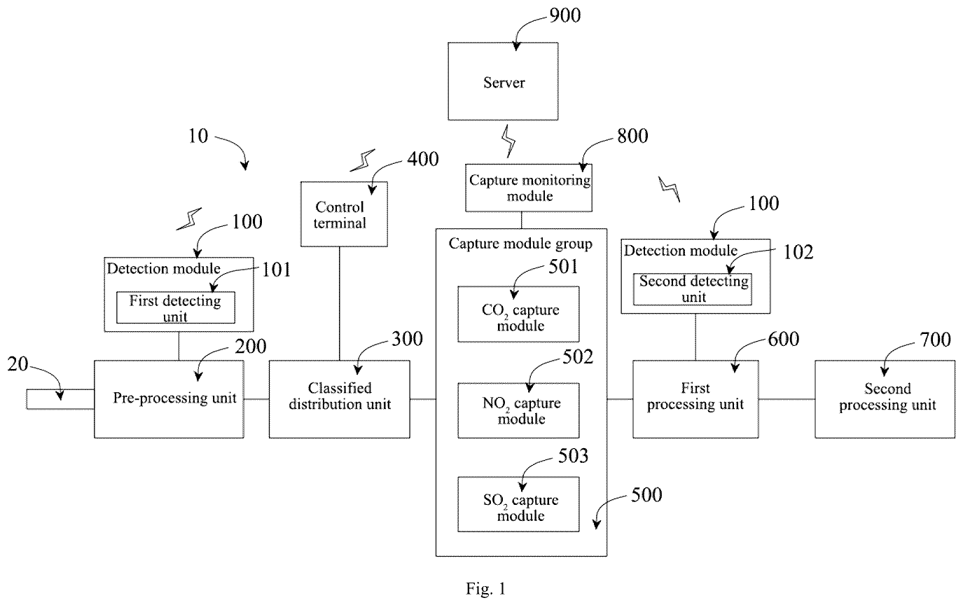

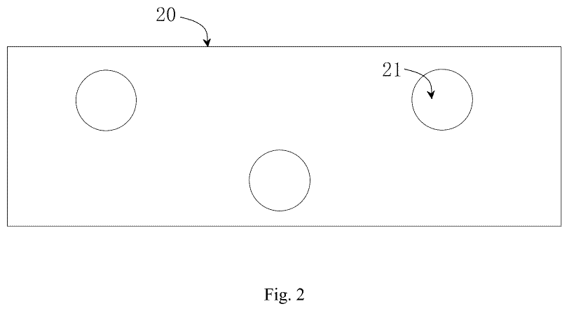

Referring to FIGS. 1-2, a capture station 10 for flue gas from chemical industrial park, includes a detection module 100, a pre-processing unit 200, a classified distribution unit 300, a control terminal 400, a capture module group 500, a first processing unit 600, a second processing unit 700, a capture monitoring module 800, and a server 900. The detection module 100 is configured to detect a flue gas. The pre-processing unit 200 is configured to be connected with a flue 20 and pre-process the flue gas. The classified distribution unit 300 is configured to for classified distribution of the flue gas. The control terminal 400 is configured for control adjustment. The capture module group 500 is configured for classified capture of the flue gas. The first processing unit 600 is configured to process a residual tail gas. The second processing unit 700 is configured to process a plurality of semi-processed products to obtain a plurality of processed products. The capture monitoring module 800 is configured to monitor the capture module group 500. And the server 900 is configured for data process and calculation.

In an embodiment, the detection module 100 includes a first detecting unit 101 and a second detecting unit 102. The first detecting unit 101 is configured to detect the flue gas in the flue 20 and the pre-processing unit 200. When detecting the flue 20, the first detecting unit 101 is arranged on a sampling hole 21 of the flue 20. The second detecting unit 102 is configured to detect the flue gas in the first processing unit 600. And the pre-processing unit 200 is configured for complete combustion and filtration of flue gas.

In an embodiment, capture module group 500 includes a carbon dioxide (CO₂) capture module 501, a nitrogen dioxide (NO₂) capture module 502 and a sulfur dioxide (SO₂) capture module 503. The classified distribution unit 300 is a flow control unit, including a plurality of conduction pipes and an electromagnetic valve, and is configured to control a flue gas flow distributed to the CO₂ capture module 501, the NO₂ capture module 502 and the SO₂ capture module 503 in the capture module group 500.

In an embodiment, the capture monitoring module 800 is configured to detect a CO₂ concentration W_{XC}, a NO₂ concentration W_{XN}, a SO₂ concentration W_{XS}, a temperature W_{XT}, a pressure W_{XP}, a flow W_{QC} distributed to the CO₂ capture module in a test capture, a flow W_{QN} distributed to the NO₂ capture module in the test capture, a flow W_{QS} distributed to the SO₂ capture module in the test capture, CO₂ capture efficiency W_{VC}, NO₂ capture efficiency W_{VN}, and SO₂ capture efficiency W_{VS} in the CO₂ capture module 501, the NO₂ capture module 502 and the SO₂ capture module 503.

In an embodiment, the server 900 can be an independent physical server, a server cluster or a distributed system consisting of multiple physical servers, or a cloud server providing basic cloud computing services, such as cloud services, cloud databases, cloud computing, cloud functions, cloud storage, network services, cloud communications, middleware services, domain name services, security services, content delivery network (CDN), and big data and artificial intelligence platforms. The server 900 is an electronic device configured to provide back-end service. For example, in this embodiment, the server 900 provides cloud computing services for the control terminal 400, particularly constructing and training a calculation model. In an embodiment, the control terminal 400 is configured to control the classified distribution unit 300 to capture and distribute. And the control terminal 400 can be an electronic device, such as a desktop computer, a laptop, a tablet computer and a smart phone, and is not limited herein.

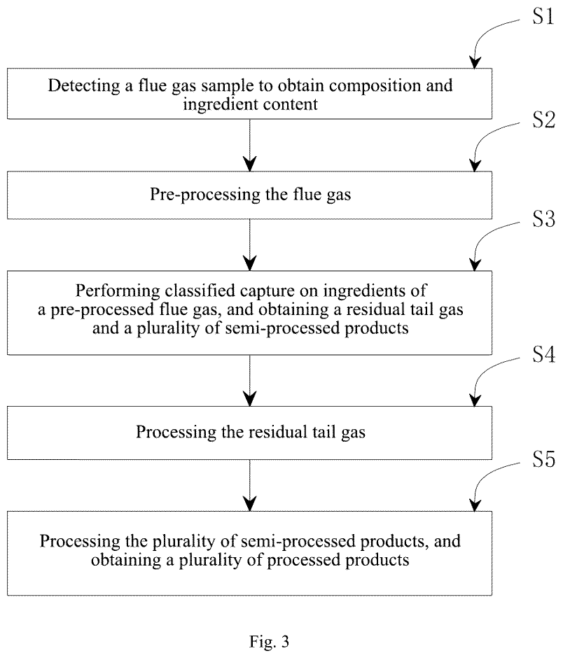

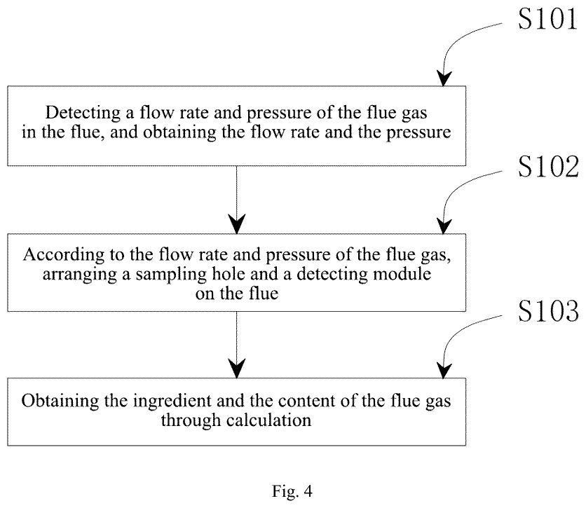

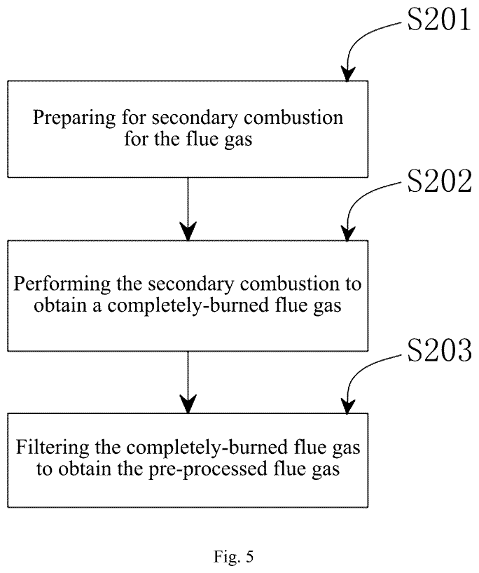

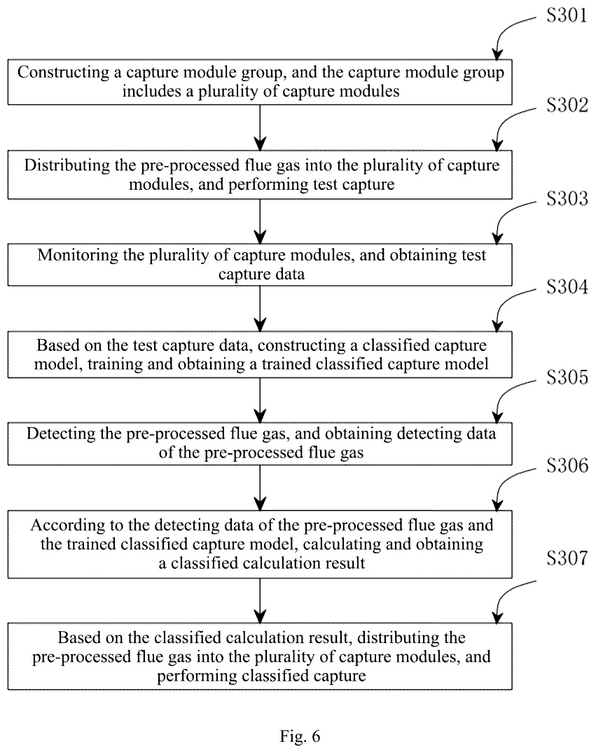

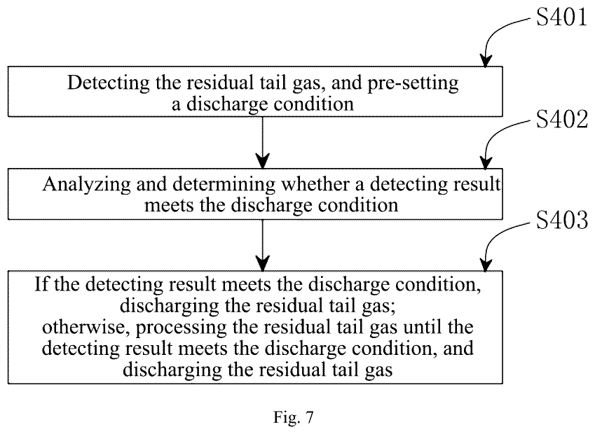

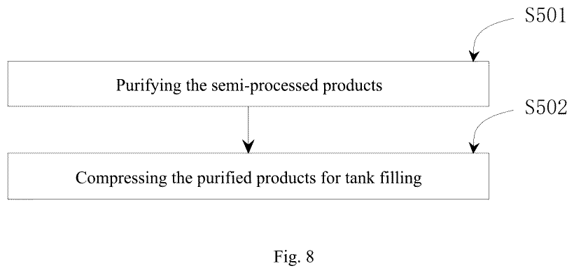

Referring to FIGS. 1-8, the present disclosure provides a capture method for flue gas from a chemical industrial park, which is performed as follows.

(S1) The flue gas sample in the flue is detected to obtain basic parameters of the flue gas sample including composition and content.

(S2) The flue gas sample is pre-processed.

(S3) Classified capture is performed on ingredients of a pre-processed flue gas to obtain a residual tail gas and a plurality of semi-processed products.

(S4) The residual tail gas is processed.

(S5) The semi-processed products are processed to obtain the processed products.

In an embodiment, the basic parameters of the flue gas sample are obtained through the following steps.

(S101) Flow rate and pressure of the flue gas sample in the flue are detected.

(S102) According to the flow rate and the pressure of the flue gas sample, the sampling hole 21 and the first detecting unit 101 are arranged on the flue.

(S103) The composition and the content of ingredients of the flue gas sample are obtained through calculation.

In an embodiment, the flue gas sample is pre-processed through the following steps.

(S201) Preparation has been made for secondary combustion of the flue gas sample, for example, an appropriate amount of oxygen is filled.

(S202) The secondary combustion is performed on the flue gas sample to obtain completely-burned flue gas.

(S203) The completely-burned flue gas is filtered to remove particulate matter, and the pre-processed flue gas is obtained.

In an embodiment, the classified capture of ingredients of the pre-processed flue gas is performed as follows.

(S301) The capture module group including the plurality of capture modules is constructed.

(S302) The pre-processed flue gas is distributed into the plurality of capture modules according to ingredient category for test capture.

(S303) The plurality of capture modules are monitored to obtain the test capture data.

(S304) Based on the test capture data, the classified capture model is further constructed and trained.

(S305) The pre-processed flue gas is analyzed to obtain relevant data.

(S306) The relevant data of the pre-processed flue gas is substituted into the trained classified capture model to obtain a classified calculation result.

(S307) Based on the classified calculation result, the pre-processed flue gas is distributed into the plurality of capture modules according to the ingredient category for classified capture.

In an embodiment, the test capture data is unifiedly collected as:

$$[ {W}_{XC} , {W}_{XN} , {W}_{XS} , {W}_{XT} , {W}_{XP} , {W}_{QC} , {W}_{QN} , {W}_{QS} , {W}_{VC} , {W}_{VN} , {W}_{VS} ] ;$$

where W_{XC} represents the CO₂ concentration; W_{XN} represents the NO₂ concentration; W_{XS} represents the SO₂ concentration; W_{XT} represents the temperature; W_{XP} represents the pressure; W_{QC} represents the flow distributed to the CO₂ capture module in the test capture; W_{QN} represents the flow distributed to the NO₂ capture module in the test capture; W_{QS} represents the flow distributed to the SO₂ capture module in the test capture; W_{VC} represents the CO₂ capture efficiency; W_{VN} represents the NO₂ capture efficiency; and W_{VS} represents the SO₂ capture efficiency.

In an embodiment, the classified capture model is expressed as follows:

$$\{ \begin{matrix}{V}_{QC} = {F}_{C} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} ) \\ {V}_{QN} = {F}_{N} ( {W}_{XC} , {W}_{XN} , {W}_{XS} , {W}_{XT} , {W}_{XP} , {W}_{QC} , \\ {W}_{QN} , {W}_{QS} , {W}_{VC} , {W}_{VN} , {W}_{VS} ) ; \\ {V}_{QS} = {F}_{S} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} )\end{matrix}$$

where V_{QC} represents a calculated flow distributed to the CO₂ capture module; V_{QN} represents a calculated flow distributed to the NO₂ capture module; and V_{QS} represents a calculated flow distributed to the SO₂ capture module;

F_{C}( ) represents a CO₂ capture-calculation model; F_{N} ( ) represents a NO₂ capture-calculation model; and FS( ) represents a SO₂ capture-calculation model.

In an embodiment, the residual tail gas is processed as follows.

(S401) The residual tail gas is detected, and a discharge condition is set.

(S402) Whether the residual tail gas meets the discharge condition is determined.

(S403) If the residual tail gas meets the discharge condition, the residual tail gas is directly discharged; otherwise, the residual tail gas is further processed through the steps (401)-(402) until the discharge condition is met, and the residual tail gas is discharged.

In an embodiment, the semi-processed products are processed through the following steps.

(S501) The semi-processed products are purified.

(S502) The purified products are compressed for tank filling.

It should be noted by those skilled in the art that an embodiment of the present disclosure can be provided as a method, a system, or a computer program product. Therefore, the present disclosure can take a form of full hardware embodiments, full software embodiments, or embodiments combining software and hardware aspects. In addition, the present disclosure can take a form of a computer program product implemented on one or multiple computer available storage media containing computer available procedure code (including but not limited to a disk memory, a compact disc read-only memory (CD-ROM) and an optical memory).

The procedure instructions can be installed on a computer or other programmable data processing equipment, thereby a series of operation steps are performed on the computer or the other programmable data processing equipment to produce computer-implemented processing, so that the procedure instructions on the computer or the other programmable data processing equipment are configured to realize one or more processes of a flow chart diagram and/or specific function steps in a one or more blocks of a block diagram. Although the preferred embodiments of the present disclosure have been described, embodiments may be subject to additional changes and modifications as long as those skilled in the art know the basic creative concepts. Therefore, the appended claims are intended to include the preferred embodiments and all changes and modifications falling within the scope of the present disclosure.

It is obvious that those skilled in the art can make various modification and transformation of the present disclosure without departing from the spirit and scope of the present invention. In this way, if the various modification and transformation of the present disclosure are within the scope of the appended claims and equivalent technical solutions of the present disclosure, the present disclosure is intended to include the various modification and transformation.

Described above are only preferred embodiments of this application, and are not intended to limit the scope of this application. Any equivalent replacements or modification made by those skilled in the art without departing from the spirit of this application shall fall within the scope of this application defined by the appended claims.

What is claimed is:

1. A capture method for flue gas from a chemical industrial park, comprising:
(S1) detecting a flue gas sample in a flue to obtain basic parameters of the flue gas sample comprising composition and content;
(S2) pre-processing the flue gas sample;
(S3) performing classified capture on ingredients of a pre-processed flue gas to obtain a residual tail gas and a plurality of semi-processed products;
(S4) processing the residual tail gas; and
(S5) processing the plurality of semi-processed products to obtain a plurality of processed products.

2. The capture method of claim 1, wherein the step of detecting a flue gas sample to obtain basic parameters of the flue gas sample comprises:
(S101) detecting and obtaining a flow rate and a pressure of the flue gas sample;
(S102) according to the flow rate and pressure of the flue gas sample, arranging a sampling hole and a detecting module on the flue; and
(S103) obtaining the composition and the content of ingredients of the flue gas sample through calculation.

3. The capture method of claim 1, wherein the step of pre-processing the flue gas sample comprises:
(S201) preparing for secondary combustion for the flue gas sample;
(S202) performing the secondary combustion on the flue gas sample to obtain a completely-burned flue gas; and
(S203) filtering the completely-burned flue gas to obtain the pre-processed flue gas.

4. The capture method of claim 1, wherein the step of performing classified capture on ingredients of a pre-processed flue gas to obtain a residual tail gas and a plurality of semi-processed products comprises:
(S301) constructing a capture module group, wherein the capture module group comprises a plurality of capture modules;
(S302) distributing the pre-processed flue gas into the plurality of capture modules according to ingredient category for test capture;
(S303) monitoring the plurality of capture modules to obtain test capture data;
(S304) based on the test capture data, constructing a classified capture model, and training the classified capture model to obtain a trained classified capture model;
(S305) analyzing the pre-processed flue gas to obtain relevant data of the pre-processed flue gas;
(S306) substituting the relevant data of the pre-processed flue gas into the trained classified capture model for calculation to obtain a classified distribution result; and
(S307) based on the classified distribution result, distributing the pre-processed flue gas into the plurality of capture modules according to the ingredient category for classified capture.

5. The capture method of claim 4, wherein the test capture data is unifiedly collected as: [W_{XC}, W_{XN}, W_{XS}, W_{XT}, W_{XP}, W_{QC}, W_{QN}, W_{QS}, W_{VC}, W_{VN}, W_{VS}];
wherein W_{XC} represents a carbon dioxide (CO₂) concentration; W_{XN} represents a nitrogen dioxide (NO₂) concentration; W_{XS} represents a sulfur dioxide (SO₂) concentration; W_{XR} represents temperature; W_{XP} represents pressure; W_{QC} represents a flow distributed to a CO₂ capture module in the test capture; W_{QN} represents a flow distributed to a NO₂ capture module in the test capture; W_{QS} represents a flow distributed to a SO₂ capture module in the test capture; W_{VC} represents CO₂ capture efficiency; W_{VN} represents NO₂ capture efficiency; and W_{VS} represents SO₂ capture efficiency.

6. The capture method of claim 5, wherein the classified capture model is expressed as follows:
$$\{ \begin{matrix}{V}_{QC} = {F}_{C} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} ) \\ {V}_{QN} = {F}_{N} ( {W}_{XC} , {W}_{XN} , {W}_{XS} , {W}_{XT} , {W}_{XP} , {W}_{QC} , \\ {W}_{QN} , {W}_{QS} , {W}_{VC} , {W}_{VN} , {W}_{VS} ) ; \\ {V}_{QS} = {F}_{S} ( {W}_{XC} , {W}_{XN} , {W}_{XS} , {W}_{XT} , {W}_{XP} , \\ {W}_{QC} , {W}_{QN} , {W}_{QS} , {W}_{VC} , {W}_{VN} , {W}_{VS} )\end{matrix}$$
wherein V_{QC} represents a calculated flow distributed to the CO₂ capture module; V_{QN} represents a calculated flow distributed to the NO₂ capture module; and V_{QS} represents a calculated flow distributed to the SO₂ capture module;
F_{C}( ) represents a CO₂ capture-calculation model; F_{N}( ) represents a NO₂ capture-calculation model; and FS ( ) represents a SO₂ capture-calculation model.

7. The capture method of claim 1, wherein the step of processing the residual tail gas comprises:
(S401) detecting the residual tail gas, and setting a discharge condition;
(S402) determining whether the residual tail gas meets the discharge condition according to a detection result; if yes, discharging the residual tail gas; otherwise, further processing the residual tail gas, and repeating steps (401)-(402) until the residual tail gas meets the discharge condition, and discharging the residual tail gas.

8. The capture method of claim 1, wherein the step of processing the plurality of semi-processed products to obtain a plurality of processed products comprises:
(S501) purifying the plurality of semi-processed products; and
(S502) compressing a plurality of purified products for tank filling.

9. A capture station for flue gas from a chemical industrial park, comprising:
a detection module;
a pre-processing unit;
a capture module group;
a classified distribution unit;
a control terminal;
a capture monitoring module;
a server;
a first processing unit; and
a second processing unit;
wherein the detection module is configured to detect a flue gas; the pre-processing unit is configured to pre-process the flue gas; the capture module group is configured for classified capture of the flue gas; the classified distribution unit is configured for classified distribution of the flue gas; the control terminal is configured to control and adjust working parameters in a whole capture process; the capture monitoring module is configured to monitor the capture module group; the server is configured for data processing and calculation; the first processing unit is configured to process a residual tail gas; and the second processing unit is configured to process a plurality of semi-processed products to obtain a plurality of processed products.
